(19) United States
(12) Patent Application Publication
(10) Pub. No.: US 2025/0012774 A1
(43) Pub. Date: Jan. 9, 2025

(54) METHODS AND SYSTEMS OF ANALYSIS OF PHOSPHORUS RELEASE AND MANAGEMENT IN A BODY OF WATER

(71) Applicant: SePRO Corporation, Carmel, IN (US)

(72) Inventors: Byran C. FUHRMANN, Rocky Mount, NC (US); Ben WILLIS, Rocky Mount, NC (US); Jason SCOTT, Whitakers, NC (US); Scott SHULER, Carmel, IN (US); Ryan VAN GOETHEM, Spokane, WA (US); Carey BARKLEY, Whitakers, NC (US)

(21) Appl. No.: 18/764,671

(22) Filed: Jul. 5, 2024

Related U.S. Application Data
(60) Provisional application No. 63/525,027, filed on Jul. 5, 2023.

(51) Int. Cl. G01N 33/18; G06F 30/27; G06F 113/08; G06F 119/22

(52) U.S. Cl. CPC G01N 33/182; G06F 30/27; G06F 2113/08; G06F 2119/22

(57) ABSTRACT

A method, system, and predictive model that incorporate various sources of unrelated sediment data and water body data to determine the rate of sediment phosphorus release. This method, system, and/or model can be fine-tuned to enhance the accuracy of predictions by providing feedback data of measured phosphorus release. This method, system, and/or model can be used to assess the effectiveness of various strategies to suppress sediment phosphorus release and these predictions can also be fine-tuned by providing feedback data of measured effectiveness of these strategies in real world treatments or laboratory studies. In one embodiment, this model includes the calculation of coefficients related to the release of different forms of sediment phosphorus to create indices of phosphorus release in a water body.

### CROSS-REFERENCE TO RELATED APPLICATIONS

This Application is related to and claims the benefit of U.S. Provisional Application No. 63/525,027, filed Jul. 5, 2024, entitled METHODS OF ANALYSIS FOR AN ADVANCED UNDERSTANDING OF SEDIMENT PHOSPHOROUS DYNAMICS AND MANAGEMENT STRATEGIES TO SUPPRESS THE RELEASE OF PHOSPHORUS INTO THE WATER COLUMN, the entirety of which is incorporated herein by reference.

### GOVERNMENT RIGHTS STATEMENT

N/A.

### TECHNICAL FIELD

This disclosure relates to a combination of analytical methods to produce a model which provides an enhanced understanding of phosphorus release from surface water sediments.

### INTRODUCTION

Sediment is a critical driver of water quality in surface waters, especially stagnant water bodies such as lakes and reservoirs. Phosphorous is the most common limiting nutrient for algae and cyanobacterial growth in surface water such as lakes, reservoirs, and rivers. Excessive phosphorus levels in the water column are associated with harmful algae blooms and excessive productivity or eutrophication. Natural surface water sediments typically contain low concentrations of phosphorus and high concentrations of phosphorus binding elements such as iron, aluminum, calcium. This allows most lake sediments to serve as a sink for phosphorus, which can be delivered to the sediment when phosphorus containing organic matter sinks and settles. However, over time, the phosphorus binding elements can become supersaturated with phosphorus and the sediment now transitions into a source of phosphorus to the water column. Anoxic conditions are often the largest source of phosphorus release from sediments, but phosphorus release can occur under a variety of environmental conditions such as disturbances, warming temperature, or fluctuations in pH. It is often important for water resource managers to determine the timing of phosphorus release and how much phosphorus is being released from the sediments in order to understanding nutrient dynamics, which drive the structure of the food web and the productivity of the water body.

Surface water management often involves the addition of chemicals to the sediment which can permanently bind the phosphorus present and prevent it from being released into the water column. Both the identification of sediment phosphorus as a large contributor to the water body eutrophication and sediment is commonly collected from eutrophic water bodies and analyzed. Sequential extractions are commonly used to determine what portion of the sediment phosphorus is potentially available to be released into the water column.

There are a variety of methods to estimate the how much phosphorus is being released from surface water sediments, but sequential extractions of different forms of sediment phosphorus is likely the most popular method. Sequential sediment phosphorus extractions are much quicker and less costly than the alternatives, such as sediment core incubations, limnocorrals, and seasonal in-lake bottom water sampling. Various sequential extractions have been developed, but they all incorporate the same general approach of starting with less chemically reactive reagent such as deionized water to pull out the most easily desorbed phosphorus species and proceeding to extract more tightly bound forms of phosphorus with more chemically reactive ingredients such as sodium hydroxide or hydrochloric acid. The masses of phosphorus in each step of the sequential extraction can be added together and this combined sum is often referred to as “potentially bioavailable phosphorus”, which means that it has the potential to be released into the water column where it can stimulate the growth of algae or cyanobacteria. A total phosphorus analysis of a comparative sample is usually performed in tandem with the sequential extraction. “Non-bioavailable phosphorus” can then be determined by subtracting potentially bioavailable phosphorus from the total phosphorus concentration.

Water body managers generally collect sediment samples from various regions within the water body to account for this variability and determine how the amount of potentially releasable phosphorus changes with GPS location. While it is helpful to have a mass of phosphorus that can potentially be released into the water column, there is a large uncertainty with how much of this phosphorus will actually be released and the timing of the release.

One-time, large sediment phosphorus treatments designed to suppress sediment phosphorus release for longer time periods (for example, five years or more) frequently fail to meet expectations due to the large uncertainty in environmental conditions over longer time frames, such as watershed loading and sediment resuspension. Adaptive management is the concept of treating sediments with phosphorus binding agents to suppress the release for one year, rather than multiple years. Adaptive management allows for a smaller upfront cost and for future treatments to be modified based on the outcomes of the prior treatment. This ultimately allows for the sediment phosphorus management strategy to be refined and optimized with each successive treatment.

Although adaptive management holds numerous advantages over one-time, large sediment phosphorus treatments, the most difficult aspect of successful implementation is obtaining an understanding of the annual phosphorus release from the sediment. Typical approaches involve laboratory sediment flux incubations to estimate release rates per area of sediment, mass balances of external and internal loading within the surface water, and temporal bottom-water phosphorus monitoring in stratified lakes and reservoirs. However, all of these approaches are time consuming, expensive, and often inaccurate.

### SUMMARY

This disclosure relates to a combination of analytical methods to produce a model which provides an enhanced understanding of phosphorus release from surface water sediments. This systematic approach to understanding short-term sediment phosphorus release allows for more accurate adaptive management strategies to suppress sediment phosphorus release to combat the growing problem of eutrophication. This model combines information from the location of the sediment sample, data from fundamental soil and/or sediment analyses, including modified parameters, and data obtained from a sediment sequential phosphorus extraction to create various coefficients that quantify fundamental properties related to the release of sediment phosphorus under various environmental conditions. The model then utilizes these coefficients to predict the portion of each sediment phosphorus fraction that is released or buried for a specific time period, based on environmental conditions and site characteristics of the location. This model can be enhanced by incorporating real-world or laboratory data to refine the sediment phosphorus release rate prediction using machine learning and/or multiple linear regressions. In addition, this method can be used to predict the efficiency of common phosphorus binding agents to suppress sediment phosphorus release, such as aluminum salts. The prediction of the efficiency of common phosphorus binding agents to suppress sediment phosphorus release can also be enhanced by incorporating real world treatment or laboratory incubation treatments to refine the predictions using machine learning and/or multiple linear regressions.

In one embodiment, a method for predicting total phosphorus release from sediment within a water body includes: obtaining water body characteristics initial input data; obtaining a sample of the sediment and obtaining, from the sample, sediment characteristics initial input data; calculating one or more coefficients and indices based on the water body characteristics initial input data and the sediment characteristics initial input data; calculating a predicted total phosphorus release value; obtaining feedback input data from the water body and/or sediment; and calculating a refined predicted total phosphorus release value based on the feedback input data.

In one aspect of the embodiment, the method further includes predicting efficacy of a method of treatment of the water body based on the water body characteristics initial input data, the sediment characteristics initial input data, and/or the one or more coefficients and indices.

In one aspect of the embodiment, the method further includes: comparing the predicted total phosphorus release value and/or the refined predicted total phosphorus release value to a threshold predictive result value; and determining if a method of treatment should be applied to the water body.

In one aspect of the embodiment, the water body characteristics initial input data and the sediment characteristics initial input data includes depth in a water column of the water body at which a sediment sample was collected below a surface of the water body, surface area of the water body, site-specific Osgood index, wet bulk density of the sediment, percent solids within the sediment, expanded dry bulk density of the sediment, compacted dry bulk density of the sediment, particle density of the sediment, expanded porosity of the sediment, compacted porosity of the sediment, labile organic matter content of the sediment, total organic matter content of the sediment, labile-to-total-organic matter ratio of the sediment; pH of the sediment, and/or annual pH range of bottom water of the water body.

In one aspect of the embodiment, the step of obtaining sediment characteristics initial input data includes performing a sediment sequential extraction to determine a total sediment phosphorus, a labile phosphorus concentration, a labile manganese concentration, a labile iron concentration, a redox-sensitive phosphorus concentration, a redox-sensitive manganese concentration, a redox-sensitive iron concentration, an organic phosphorus concentration, a metal-oxide phosphorus concentration, a metal-oxide iron concentration, a metal-oxide aluminum concentration, an aluminum-to-phosphorus ratio, a carbon-to-phosphorus ratio, an alkaline-insoluble, acid-soluble phosphorus concentration, an alkaline-insoluble, acid-soluble calcium concentration, an alkaline-insoluble, acid-soluble lanthanum concentration, an alkaline-insoluble-metal-to-phosphorus ratio, and/or a residual phosphorus concentration.

In one embodiment, a computer-implemented method for predicting total phosphorus release from sediment within a water body includes: transmitting, to a computer, water body characteristics initial input data and sediment characteristics initial input data; performing sediment sequential extraction on a sample of the sediment from within the water body to obtain sediment extraction initial input data; calculating, with processing circuitry of the computer, at least one sediment characteristic coefficient using the water body characteristics initial input data, the sediment characteristics initial input data, and/or the sediment extraction initial input data; calculating, with the processing circuitry of the computer, at least one water column release index using the water body characteristics initial input data, the sediment characteristics initial input data, the sediment extraction initial input data, and/or the at least one sediment characteristic coefficient; calculating, with the processing circuitry of the computer, a total water column release index using the at least one water column release index; calculating, with the processing circuitry of the computer, a predicted total phosphorus release value; transmitting to a computer feedback input data, the feedback input data including updated water body characteristics initial input data, updated sediment characteristics initial input data, and/or updated sediment extraction initial input data; and calculating a refined predicted total phosphorus release value based on the feedback input data.

In one aspect of the embodiment, the method further includes: comparing, with the processing circuitry of the computer, the predicted total phosphorus release value and/or the refined predicted total phosphorus release value to a threshold predictive result value and determining if a method of treatment should be applied to the water body based on the comparison.

In one aspect of the embodiment, the method further includes: calculating, with the processing circuitry of the computer, a predicted aluminum binding efficiency value based on sediment.

In one aspect of the embodiment, the predicted aluminum binding efficiency value is calculated based on an aluminum-to-phosphorus ratio, a metal oxide phosphorus concentration, a sediment pH, a depth in the water column at which the sediment sample was collected below a surface of the water body, and/or an annual pH range of bottom water of the water body.

In one aspect of the embodiment, the method of treatment is an application of aluminum salts to the water body, the determination whether the method of treatment should be applied to the water body being based on the predicted aluminum binding efficiency value.

In one aspect of the embodiment, the feedback input data includes updated water body characteristics initial input data, updated sediment characteristics initial input data, and/or updated sediment extraction initial input data that are measured after application of a method of treatment to the water body.

In one aspect of the embodiment, the feedback input data includes updated water body characteristics initial input data, updated sediment characteristics initial input data, and/or updated sediment extraction initial input data that are measured from sediment incubations, mesocosms, limnocorrals, and/or real-word water column phosphorus concentrations.

In one aspect of the embodiment, the water body characteristics initial input data include depth in a water column at which the sediment sample was collected below a surface of the water body, a surface area of the water body, a site-specific Osgood index, and/or annual pH range of bottom water.

In one aspect of the embodiment, the sediment characteristics initial input data include wet bulk density, percent solids, expanded dry bulk density, compacted dry bulk density, particle density, expanded porosity, compacted porosity, labile organic matter content, total organic matter content, labile-to-organic matter ratio, and/or pH.

In one aspect of the embodiment, the sediment extraction initial input data include a total sediment phosphorus, a labile phosphorus concentration, a labile manganese concentration, a labile iron concentration, a redox-sensitive phosphorus concentration, a redox-sensitive manganese concentration, a redox-sensitive iron concentration, a redox-sensitive-iron-to-redox-sensitive-phosphorus ratio, an organic phosphorus concentration, a metal-oxide phosphorus concentration, a metal-oxide iron concentration, a metal-oxide aluminum concentration, aluminum-to-phosphorus ratio, a carbon-to-phosphorus ratio, an alkaline-insoluble, acid-soluble phosphorus concentration, an alkaline-insoluble, acid-soluble calcium concentration, an alkaline-insoluble, acid-soluble lanthanum concentration, an alkaline-insoluble-metal-to-phosphorus ratio, and/or a residual phosphorus concentration.

In one aspect of the embodiment, the at least one sediment characteristic coefficient includes a sediment expansion coefficient, a sediment disturbance coefficient, and/or an iron stripping coefficient.

In one aspect of the embodiment, the sediment expansion coefficient is calculated by the processing circuitry of the computer according to the equation:

$$\frac{Compacted dry bulk density ( \frac{g}{{cm}^{3}} )}{Expanded dry bulk density ( \frac{g}{{cm}^{3}} )} = \frac{\frac{mass of dry particles ( g )}{volume of dry particles ( {cm}^{3} )}}{Wet bulk density ( \frac{g}{{cm}^{3}} ) \times \% solids}$$

In one aspect of the embodiment, the at least one water column release initial index includes an oxygen depletion index, a diffusion index, a redox release index, and/or an organic phosphorus release index.

In one embodiment, a model for predicting total phosphorus release from sediment within a water body includes: providing a database of water body characteristics initial input data obtained from a water body, a database of sediment characteristics initial input data obtained from sediment from within the water body, and a database of sediment sequential extraction initial input data obtained from sequential extraction of a sample of the sediment from within the water body; calculating at least one sediment characteristic coefficient using the water body characteristics initial input data, the sediment characteristics initial input data, and/or the sediment extraction initial input data; calculating at least one water column release initial index using the water body characteristics initial input data, the sediment characteristics initial input data, the sediment extraction initial input data, and/or the at least one sediment characteristic coefficient; calculating a total water column release index using the at least one water column release index; calculating a predicted total phosphorus release value; obtaining feedback input data from the water body and sediment, the feedback input data including updated water body characteristics initial input data, updated sediment characteristics initial input data, and/or updated sediment extraction initial input data; and calculating a refined predicted total phosphorus release value based on the feedback input data.

In one aspect of the embodiment, the model further includes calculating a predicted aluminum binding efficiency value based on the water body characteristics initial input data, the sediment characteristics initial input data, the sediment extraction initial input data, and/or the feedback input data.

### BRIEF DESCRIPTION OF THE DRAWINGS

A more complete understanding of embodiments described herein, and the attendant advantages and features thereof, will be more readily understood by reference to the following detailed description when considered in conjunction with the accompanying drawings wherein:

FIG. 1 is a flow diagram showing a general method of predictively analyzing phosphorus release characteristics of a water body, in accordance with the present disclosure;

FIG. 2 is a flow diagram showing a method of predictively analyzing phosphorus release characteristics of a water body, in accordance with the present disclosure;

FIG. 3 is a flow diagram showing the relationships between measured parameters (site information, sediment sequential extraction data, and sediment properties), calculated indices and coefficients, a water column release index, and a predicted phosphorus release over time, in accordance with the present disclosure;

FIG. 4 is a flow diagram showing interconnected data for the predictive sediment phosphorus release model of FIG. 3 and the feedback data pathways to enhance the model's future predictive accuracy, in accordance with the present disclosure;

FIG. 5 is a stylized diagram of an exemplary system for performing the methods discussed herein, in accordance with the present disclosure;

FIG. 6 is a chart showing the correlation between the sediment expansion coefficient and sediment porosity, percent organic matter, and percent solids;

FIG. 7 is a chart showing the correlation between the percentage of the total organic matter content that is labile (lost at a temperature of 250° C.) and the percentage of plant or algae phosphorus that is easily extractable with a high speed blender and 1 M ammonium chloride extraction; and

FIG. 8 is a chart showing how the aluminum-to-phosphorus ratio of natural sediments is substantially different within different lakes and at different depths within the same lake.

### DETAILED DESCRIPTION

Some embodiments advantageously provide a deeper understanding of the potential of surface water sediments to release phosphorus into the overlaying water column, as well as to classify the risk of phosphorus release based on a variety of sediment parameters which are compared amongst the values within a large database of previous sediment samples. In some embodiments, system is configured to build upon itself, with more sediment analyses leading to more informative and accurate predictions of the risk of phosphorus release. Additionally, measured phosphorus release from sediment incubations, mesocosms, limnocorrals, and/or real-world water column phosphorus concentrations can be used to provide feedback data to improve the accuracy of the relationships between parameters, calculated coefficients, indexes, and the predicted phosphorus release over time. In some embodiments, a method is disclosed for predicting total phosphorus release from a sediment within a water body and/or a method for recommending a method of treatment of the water body (for example, a method or system of binding and/or removing phosphorus and/or other nutrients, and/or a method of controlling aquatic algae and/or plants that release nutrients) and/or a method for predicting the efficacy of a particular method of treatment of the water body. Collectively, these methods are referred to herein for simplicity as a method of predicting total phosphorus release, even though they may also include remediation methods. In some embodiments, this/these methods are embodied in a predictive model that may be used to predict a potential of surface water sediments to release phosphorus into the overlaying water column, as well as to classify the risk of phosphorus release based on a variety of sediment parameters which are compared amongst the values within a large database of previous sediment samples. In some embodiments, this/these methods are computer-implemented method(s). In some embodiments, this/these method(s) and/or predictive model are embodied in software that is coded and/or programmed to perform the method(s) discussed herein.

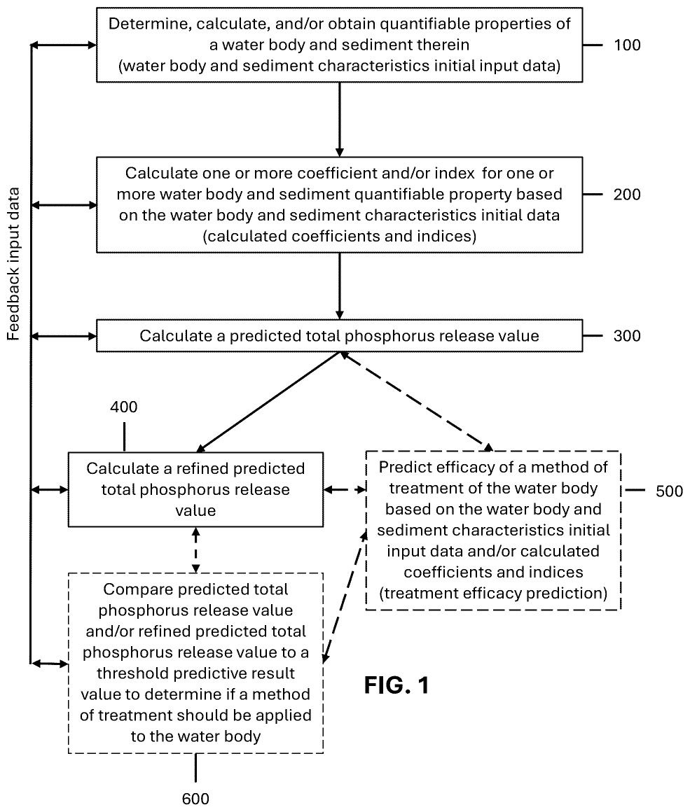

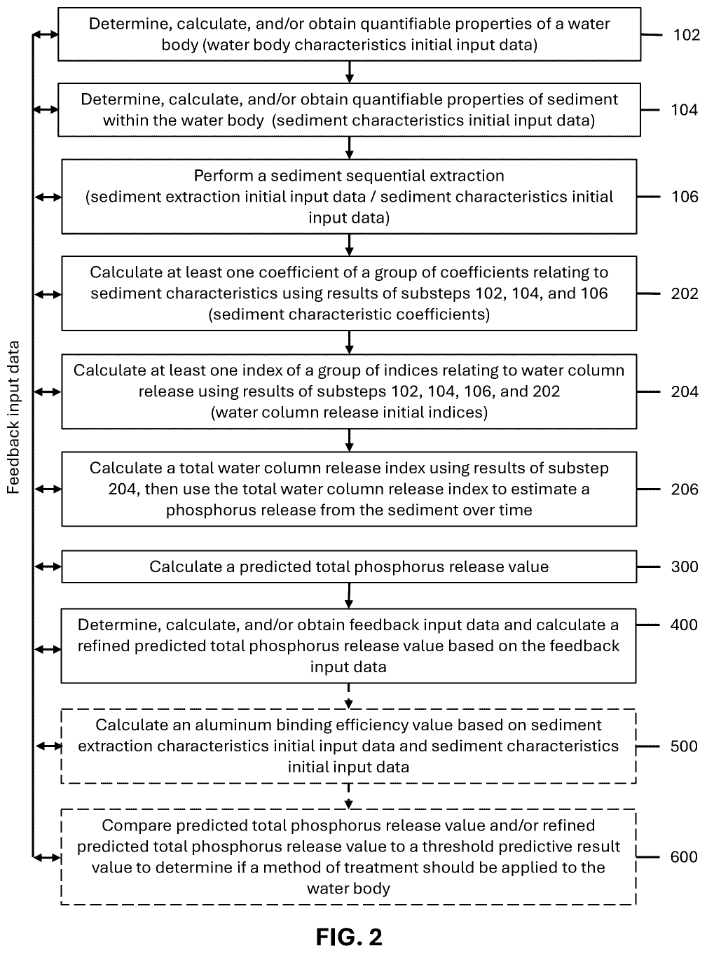

Referring now to FIG. 1, a method of predictively analyzing phosphorus release characteristics of a water body is shown generally. In one embodiment, the method generally includes gathering, determining, calculating, and/or otherwise obtaining quantifiable properties of the water body and sediment therein (referred to herein as water body and sediment characteristics initial input data), using that information to calculate one or more coefficients and/or indices for one or more water body and sediment characteristic, then using those coefficients and/or indices to generate a predictive result. In one embodiment, the predictive result is a predicted total phosphorus release value. In some embodiments, the predictive result is used to determine whether a treatment of the water body is recommended and, if so, what treatment is appropriate. For example, the predictive result may be compared to a threshold predictive result value that indicates when a water body may warrant binding and/or removal of phosphorus, or other treatment to help prevent anoxic conditions. As noted above, the method may be continually, periodically, randomly, and/or manually (at will) updated with real-time or other data to refine and enhance the accuracy of the prediction outcome. Further, one or more of the general steps shown in FIG. 1 may comprise one or more substeps, such as is as shown in FIG. 2. The steps shown in the figures and described herein constitute a method or algorithm; however, in some embodiments, performance of the steps is embodied in a model that may be adjusted and updated with new or supplemental data.

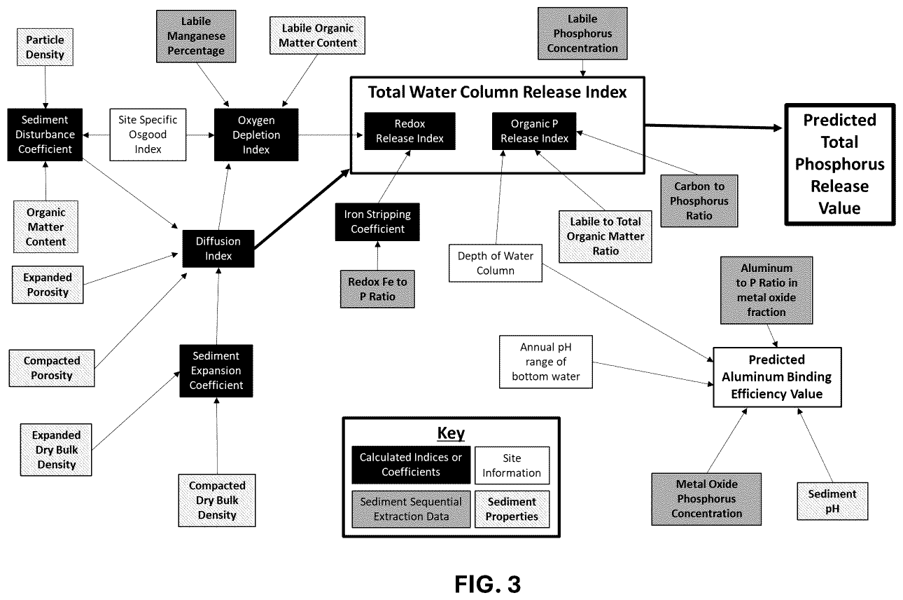

Continuing to refer to FIG. 1, in an exemplary first step 100 one or more quantifiable properties of a water body and sediment therein are determined, calculated, and/or otherwise obtained. As shown in FIG. 3, these quantifiable properties are identified as site information (white boxes without shading), sediment properties (boxes shaded in light gray), and sediment sequential extraction data (boxes shaded in darker gray). These results are referred to herein and shown in FIG. 1 as water body and sediment characteristics initial input data. In some embodiments, the water body and sediment characteristics initial input data include, but are not limited to: depth of the sediment sample below the water surface; area of the water body; site-specific Osgood index; wet bulk density of sediment; percent solids in the sediment; expanded dry bulk density of the sediment; compacted dry bulk density of the sediment; particle density of the sediment; expanded porosity of the sediment; compacted porosity of the sediment; labile organic matter content in the sediment; total organic matter in the sediment; labile-to-organic matter ratio of the sediment; pH of the sediment; the annual pH range of the bottom water (within 1-3 feet of the sediment); total sediment phosphorus; concentrations of labile phosphorus, labile, manganese, and labile iron; concentrations of redox-sensitive phosphorus, manganese, and iron; concentrations of organic phosphorus, metal-oxide phosphorus, metal-oxide iron, and metal-oxide aluminum; concentrations of alkaline-insoluble, acid-soluble phosphorus, calcium, and lanthanum; and/or concentration of residual phosphorus.

Continuing to refer to FIG. 1, in an exemplary second step 200 one or more coefficients and/or indices for one or more water body and sediment quantifiable properties (referred to herein as “calculated coefficients and indices”) are calculated based on the water body and sediment characteristics initial input data determined in the first step 100. These calculated coefficients and indices are shown in the black boxes in FIG. 3. In some embodiments (for example, as shown in FIG. 3), the one or more coefficients and/or indices include, but are not limited to: sediment expansion coefficient; sediment disturbance coefficient; diffusive release coefficient; iron stripping coefficient; oxygen depletion index; diffusion index; redox release index; organic phosphorus release index; total water column release index; and/or an estimation of aluminum binding efficiency of aluminum salts. In one embodiment, the total water column release index is calculated based on the redox release index and the organic phosphorus release index, and on all the other initial input data and calculated coefficients and indices, either directly or indirectly. In one embodiment, the total water column release index is used to calculate the predicted total phosphorus release value.

Continuing to refer to FIG. 1, in an exemplary third step 300 a predicted total phosphorus release value is calculated. In one embodiment, the predicted total phosphorus release value is a predicted total phosphorus release value over time.

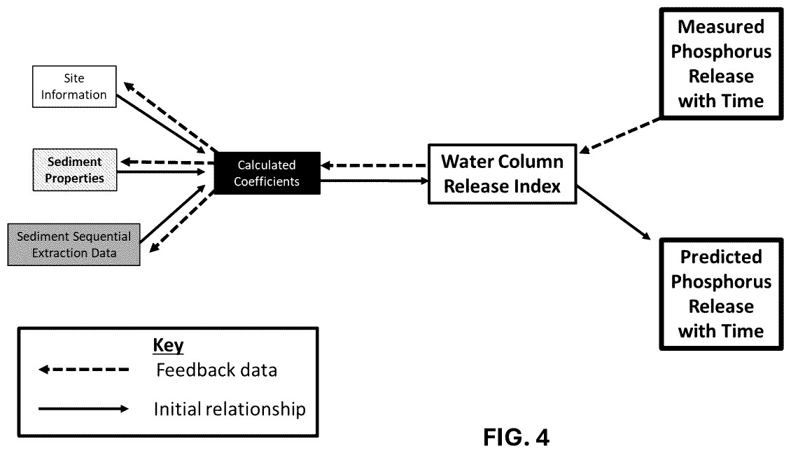

Continuing to refer to FIG. 1, in an exemplary fourth step 400 a refined predicted total phosphorus release value is calculated. In one embodiment, the fourth step 400 includes determining, calculating, and/or obtaining feedback input data. For example, in one embodiment, the refined predicted total phosphorus release value is calculated by inputting feedback data and/or additional or updated water body and sediment characteristics initial input data (collectively, referred to herein as feedback input data). In some embodiments, the coefficients and/or indices calculated in step 200 are updated or modified based in the entry of the feedback input data. Feedback input data may be entered in real time as it is obtained (manually, automatically, and/or semi-automatically), may be entered at a fixed schedule (for example, hourly, daily, weekly, or monthly, or at pre-set intervals), may be entered randomly, and/or at other pre-determined or unknown times or time intervals. As shown in FIGS. 1 and 2, the feedback input data may be acquired and/or entered into the algorithm or model at any step of the method, and the other calculations may be subsequently adjusted accordingly. In one non-limiting example, the model may suggest or recommend a method of treatment of the water body to suppress the release of nutrients, such as phosphorus. If a treatment method is applied (regardless of whether a recommendation is made), the method may include resampling the water body and/or sediment after treatment to obtain feedback input data to refine the predicted total phosphorus release value, the predicted aluminum binding efficiency, and/or other metrics of the model (for example, sediment phosphorus release may be sampled to determine if suppression has occurred). The method(s) of treatment may be any method of treatment used in water management, including, but not limited to, phosphorus management strategies. The refined predicted total phosphorus release value is calculated once or many times during performance of the method. Thus, this step establishes a feedback loop within the system that refines predictions and accuracy (for example, as shown in FIG. 4).

Continuing to refer to FIG. 1, an optional fifth step 500 is shown. In this optional exemplary fifth step 500, the efficacy or efficiency of a suggested method of treatment of the water body is predicted (referred to herein as “treatment efficacy prediction”). In one embodiment, this prediction is based on the water body and/or sediment characteristics initial input data (for example, from the first step 100 of the method) and/or the calculated coefficients and/or indices (for example, from the second step 200) of the method. This treatment efficacy prediction may be used to evaluate one or more proposed treatments to determine which one(s) would be the most effective in binding and/or removing phosphorus from the water body, based on the properties of that water body and its sediment. Additionally or alternatively, the treatment efficacy prediction may be used as a mitigating or exacerbating factor in the determination of the predicted total phosphorus release value and/or the refined predicted total phosphorus release value. Although this optional fifth step 500 is shown at the end of the method, in some embodiments this is a standalone optional step that may be conducted at any stage of the method, either in parallel or as part of one or more other steps, such as in the first and/or second steps 100, 200.

Continuing to refer to FIG. 1, an optional sixth step 600 is shown. In this optional exemplary sixth step 600, the predicted total phosphorus release value and/or the refined predicted total phosphorus release value are compared to a threshold predictive result value that indicates when a method of treatment should be applied to the water body. For example, the comparison may indicate binding and/or removal of phosphorus should be performed, or other treatment to help prevent anoxic conditions. For example, if the predicted total phosphorus release value and/or the refined predicted total phosphorus release value are higher than a threshold associated value, such result may indicate to an operator that preventative and/or remedial action is recommended. Further, in some embodiments, if the comparison suggests a method of treatment should be performed, the efficacy of one or more methods of treatment may be evaluated using the optional fifth step 500 discussed above to determine the best option. The optional sixth step 600 may be performed before or after the optional fifth step 500, if the optional sixth step 600 is performed.

Referring now to FIG. 2, an exemplary method of predictively analyzing phosphorus release characteristics of a water body is shown with substeps to the general steps shown in FIG. 1. It will be understood that, in some embodiments, the substeps shown in FIG. 2 may be performed in a different order and/or more or fewer substeps than those shown may be performed. However, not all general steps include substeps; for example, in some embodiments the third and fourth general steps 300, 400 are single-process steps.

Continuing to refer to FIG. 2, in one embodiment, the first general step 100 of FIG. 1 (determination, calculation, and/or obtention of one or more quantifiable properties of a water body and sediment therein) includes a first substep 102, a second substep 104, and a third substep 106. In one embodiment, in the first substep 102 (or a first overall step) quantifiable properties about the water body being evaluated are determined, calculated, and/or otherwise obtained (referred to herein as “water body characteristics initial input data”). The water body characteristics initial input data may be directly measured and/or determined or calculated using various known means. In some embodiments, the water body characteristics initial input data that may be obtained in this substep include, but are not limited to:
a. Depth in the water column at which the sediment sample was collected below the surface of the water body;
b. Surface area of the water body;
c. Site-specific Osgood index, calculated by the depth at which the sediment sample was taken (in meters) divided by the square root of the surface area of the water body (in km²);
d. Annual pH range of the bottom water. In one embodiment, this is measured with a water quality sonde (probe) at various time intervals throughout the year, such as hourly, daily, weekly, monthly, and/or quarterly.

Continuing to refer to FIG. 2 and the first substep 102, in some embodiments, the depth of the water column is related to the releasability of organic phosphorus in the sediment. Sediments from more shallow depths have more degradable organic matter, which contains more releasable phosphorus. Sediments from shallow depths also experience higher temperatures than sediments from deeper depths, entailing a higher microbiologic metabolism and demand for carbon and phosphorus, ultimately resulting in greater degradation of organic matter. In some embodiments, the calculations utilizing data relating to the depth in the water column below the surface of the water body at which the sediment sample was collected will also take into account the climate of the water body. For example, a shallow depth in a temperate lake may be the same temperature as a deeper depth in a tropical lake.

Continuing to refer to FIG. 2 and the first substep 102, in some embodiments, the site-specific Osgood index is used to predict the potential for oxygen-rich surface water to replenish dissolved oxygen that is depleted in the bottom water near the sediment-water interface. A higher site-specific Osgood index (for example, >7) entails less mixing of oxygen-rich surface water and a higher potential for anoxia if all other factors are equal.

Continuing to refer to FIG. 2 and the first substep 102 of the first general step 100, in one embodiment, the annual pH range of the bottom water may be compared to an optimal range for the binding of aluminum to phosphorus, to help identify any seasonal windows when the pH may be outside of the optimal range. This may provide insight into the best timing to add aluminum salt while minimizing dissolved aluminum and aluminum-bound phosphorus release.

Continuing to refer to FIG. 2, in one embodiment in the second substep 104 (or a second overall step), quantifiable properties about the sediment within the water body being evaluated are determined, calculated, and/or otherwise obtained (referred to herein as “sediment characteristics initial input data”). The sediment characteristics initial input data may be directly measured and/or determined or calculated using various known means. In some embodiments, the sediment characteristics initial input data that may be obtained in this substep include, but are not limited to:
a. Wet bulk density of the sediment. In one embodiment, this is based on a determination of the mass of wet sediment that occupies a known volume.
b. Percent solids within the sediment. In one embodiment, this is determined based on the percentage of mass that remains after a known mass of wet sediment is evaporated at up to 105° C., in a desiccator, or using an alternative method to remove all water molecules from the sediment.
c. Expanded dry bulk density of the sediment. In one embodiment, this is determined by multiplying the wet bulk density by the percent solids of a sediment sample.
d. Compacted dry bulk density of the sediment. In one embodiment, this is determined based on the volume of space occupied by a known mass of dry sediment.
e. Particle density of the sediment. In one embodiment, this is determined by dividing the mass of the dry sediment by the water volume displacement when it is added to a known volume of water.
f. Expanded porosity of the sediment. In one embodiment, this is determined by dividing the expanded dry bulk density by the particle density.
g. Compacted porosity of the sediment. In one embodiment, this is determined by dividing the compacted dry bulk density by the particle density.
h. Labile organic matter content of the sediment, determined by the loss on ignition of dried sediment that is heated to 250° C. for 24 hours.
i. Total organic matter content of the sediment. In one embodiment, this is determined based on the loss on ignition of dried sediment that is heated to 500° C. for 24 hours.
j. Labile-to-total-organic matter ratio of the sediment. In one embodiment, this is determined by dividing the labile organic matter content by the total organic matter content.
k. pH of the sediment. In one embodiment, this is measured using a pH probe and a 0.02 M calcium chloride extraction, using approximately 1 part sediment to 1 part deionized water, with a 0.01 M total calcium chloride concentration in the solution.

Continuing to refer to FIG. 2 and the second substep 104 of the first general step 100, in one embodiment, the expanded dry bulk density provides information about the mass of particles in a given volume of the surficial sediment which is present at the sediment-water interface. A low dry bulk density (for example, <0.2 g/cm³) generally demonstrates a stronger interaction between organic matter and clay, which ultimately increases the distance between sediment particles. This expansion of the sediment allows it to be disturbed more easily and for the diffusion of contaminants like phosphorus to be released more easily.

Continuing to refer to FIG. 2 and the second substep 104 of the first general step 100, in one embodiment, the compacted dry bulk density provides information about the compressibility of the sediment, because it removes the influence of the water molecules that allow for the repulsion of organic matter and clay particles, which ultimately expands the sediment. Therefore, the compacted dry bulk density provides information about sediment properties below the sediment-water interface when compaction from overlaying sediment squeezes water out of the pores.

Continuing to refer to FIG. 2 and the second substep 104 of the first general step 100, in one embodiment, the particle density of the sediment is used in the method to adjust the depth of the sediment that can interact with the overlaying water column and the extent of the interaction where sediment particles are dispersed in the water column and labile phosphorus diffused into the water. Larger particle densities (for example, >2.2 g/cm³) are more resistant to disturbance and settle faster, whereas smaller particle densities (for example, 1.5 g/cm³) are more easily disturbed and remain in the water column longer, ultimately allowing for the diffusion of more labile phosphorus into the water column.

Continuing to refer to FIG. 2 and the second substep 104 of the first general step 100, in one embodiment, in one embodiment labile organic matter content of the sediment is determined by the loss on ignition of dried sediment that is heated to 250° C. for 24 hours, as noted above. Heating at a low temperature prevents the loss of high molecular weight organic compounds that are poorly degradable or completely undegradable. Some of these compounds may include humic and fulvic acids (humic substances), which are considered to be the most common form of organic matter in lake sediments, but almost completely undegradable. In some embodiments, utilizing this metric in the method ultimately may provide a better understanding of the influence of the organic matter on the sediment oxygen demand than total organic matter.

Continuing to refer to FIG. 2 and the second substep 104 of the first general step 100, in one embodiment, total organic matter content is used in the method to adjust the residence time in the water column after disturbance, with high organic matter content leading to a longer residence time due to the hydrophobic interactions with the water and the organic-rich sediment that deters settling.

Continuing to refer to FIG. 2 and the second substep 104 of the first general step 100, in one embodiment, the labile-to-total-organic matter ratio of the sediment is used in the method to provide an understanding of the extent of degradation of the organic matter present or the “freshness” of the organic matter. A high ratio (for example, >50%) of labile organic matter suggests that the organic matter came from a recently deceased organism and more extensive degradation will occur, including the depletion of oxygen and the release or organic phosphorus. A high ratio of labile organic matter to total organic matter also indicates that management activities that reduce the growth of aquatic plants and/or algae can result in an improvement in sediment physical properties such as an increase in expanded dry bulk density and porosity, because the lack of new organic matter deposition and the degradation of the fresh organic matter will significantly reduce the organic matter content of the sediment. A low ratio (<30%) of the labile organic matter suggest that the majority of the bioavailable organic matter has already been decomposed and the legacy organic matter left is poorly degradable. This, in turn, suggest not only a lower potential for oxygen depletion and organic phosphorus release, but also a lower potential for the improvement of physical sediment characteristics with aquatic plant and/or algae management. In some embodiments, the predictive ability of the algorithm used in this method regarding change in sediment physical properties may be enhanced when sediment physical properties are measured after management activities and/or lab experiments that simulate management activities (for example, feedback input data).

Continuing to refer to FIG. 2, in one embodiment in the third substep 106 (or a third overall step) a sediment sequential extraction is performed (referred to herein as “sediment extraction characteristics initial input data,” which falls within sediment characteristics initial input data). In one embodiment, the values determined in this third substep 106 include, but are not limited to:
a. The total sediment phosphorus. In one embodiment, this is determined using an acidic persulfate digestion method to convert all sediment phosphorus into the orthophosphate form, which is measured using, for example, spectrophotometry or inductively coupled plasma (ICP) spectroscopy.
b. The labile phosphorus concentration, the labile manganese concentration, and/or the labile iron concentration. In one embodiment, these are determined by the addition of a salt solution (such as ammonium chloride or potassium chloride) to release loosely bound phosphate, manganese, and/or iron from the porewater (for example, water that is found in the small spaces between sediment particles) into the extractant solution, which is measured using, for example, ICP spectroscopy.
c. The redox-sensitive phosphorus concentration, the redox-sensitive manganese concentration, and/or the redox-sensitive iron concentration. In one embodiment, these are determined by the addition of 0.11 M dithionite buffered with 0.11 M sodium bicarbonate to release redox-sensitive phosphate, manganese, and/or iron from the sediment into the extractant solution, which is measured using, for example, ICP spectroscopy.
   i. Additionally, in one embodiment the labile manganese percentage is calculated by dividing the concentration of the labile manganese by the sum of the labile and redox-sensitive manganese.
   ii. Additionally, in one embodiment a redox-sensitive-iron-to-redox-sensitive-phosphorus ratio is calculated by dividing the sum of the labile and redox-sensitive iron by the sum of the labile and redox-sensitive phosphorus.
d. The organic phosphorus concentration and metal-oxide phosphorus concentration, the metal-oxide iron concentration, and/or the metal-oxide aluminum concentration. In one embodiment, these are determined by the addition of a strong base (such as 0.1 M sodium or potassium hydroxide) to release pH-sensitive phosphate, pH-sensitive iron, and/or pH-sensitive aluminum from the sediment into the extractant solution.
   i. In one embodiment, the metal-oxide phosphorus concentration is determined using a spectrophotometric reaction specific to phosphate.
   ii. Additionally, in one embodiment the total phosphorus, total metal-oxide iron, and/or total metal-oxide aluminum are determined using ICP spectroscopy.
   iii. Additionally, in one embodiment the organic phosphorus concentration is calculated by subtracting the metal-oxide phosphorus concentration from the total phosphorus.
   iv. Additionally, in one embodiment an aluminum-to-phosphorus ratio is calculated by dividing the sum of the moles of aluminum by the moles of the metal-oxide phosphorus.
   v. Additionally, in one embodiment a carbon-to-phosphorus ratio is determined by assuming that 58% of the organic matter is carbon and dividing the stoichiometric carbon content (moles of carbon per kg dry sediment) by the organic phosphorus concentration (moles of phosphorus per kg dry sediment).
e. The alkaline-insoluble, acid-soluble phosphorus concentration, the alkaline-insoluble, acid-soluble calcium concentration, and/or the alkaline-insoluble, acid-soluble lanthanum concentration. In one embodiment, these are determined by the addition of a strong acid (such as 1 M HCl) to release highly stable phosphorus minerals, such as calcium and lanthanum phosphates, from the sediment into the extractant solution, which is measured using, for example, ICP spectroscopy.
   i. Additionally, in one embodiment an alkaline-insoluble-metal-to-phosphorus ratio is calculated by dividing the sum of the moles of calcium and lanthanum by the moles of the alkaline-insoluble phosphorus.
f. The residual phosphorus concentration. In one embodiment, this is determined by subtracting the labile, redox-sensitive, metal oxide, organic, and alkaline-insoluble phosphorus from the total phosphorus.

Continuing to refer to FIG. 2 and to the third substep 108 of the first general step 100, in one embodiment, the total sediment phosphorus is used in the method in a comparison of the total phosphorus concentration of the sediment in various sites within the same water body to predict the accumulation or enrichment of phosphorus in certain zones, which can help illustrate mechanisms of phosphorus transport within the water body. For example, phosphorus accumulation is frequent in deeper zones (for example, adjacent dams in reservoirs), in shallow coves and bays where algae accumulate and die, and/or in mid-depth zones where oxic-anoxic transitions occur more frequently and larger microbiological biofilms can form, which prevent phosphorus-containing organic matter from continuing to slowly migrate toward deeper depths. Identification of locations of zones in which phosphorus preferentially accumulates within the water body can be used to inform management strategies to intercept the phosphorus proactively, such as by treating algae and/or aquatic plants that might utilize the phosphorus for excessive growth.

Continuing to refer to FIG. 2 and to the third substep 108 of the first general step 100, in one embodiment, the labile manganese percentage is calculated in the method by dividing the concentration of the labile manganese by the sum of the labile and redox-sensitive manganese, as noted above. Dissolved manganese does not oxidize as quickly as dissolved iron typically does, and it can therefore be used as a conservative tracer of anoxic conditions. In one embodiment, a high percentage (for example, >approximately 60%) of labile manganese calculated in the method indicates that the sediment has recently been anoxic, whereas a low percentage (for example, approximately <30%) of labile manganese indicates that the sediment-water interface has been mostly oxic in the past few months. In some embodiments, the timing of the sediment collection is also considered when performing the method, as a lower percentage of labile manganese will be expected to be present during cooler conditions and a higher percentage of labile manganese will be expected to be present in warmer conditions (for example, the summer and fall.)

Continuing to refer to FIG. 2 and to the third substep 108 of the first general step 100, in one embodiment, the redox-sensitive-iron-to-redox-sensitive-phosphorus ratio is used in the method because it indicates whether iron-binding sites are supersaturated (for example, an iron-to-phosphorus ratio of <10) and/or if there is excessive iron, which could prevent the release of phosphorus even after reductive dissolution, when both ins encounter dissolved oxygen (for example, an iron-to-phosphorus ratio of >20).

Continuing to refer to FIG. 2 and to the third substep 108 of the first general step 100, in one embodiment, the aluminum-to-phosphorus ratio is used in the method because it indicates a general understanding of the expected aluminum-to-phosphorus binding ratio that would result from the addition of aluminum salt to suppress phosphorus release.

Continuing to refer to FIG. 2 and to the third substep 108 of the first general step 100, in one embodiment, the carbon-to-phosphorus ratio is used in parallel with the other metrics (including, but not limited to, the depth of the water column above the sediment and the ratio of the labile organic matter to total organic matter), which indicates the biodegradability of the sediment organic matter. The algorithm of the method may also be adapted to incorporate an understanding of the oxygen depletion potential of the sediment and to calculate the expected conversion of organic phosphorus to polyphosphate under oxic conditions and the expected conversion of organic phosphorus and polyphosphates under anaerobic conditions.

Continuing to refer to FIG. 2 and to the third substep 108 of the first general step 100, in one embodiment, the alkaline-insoluble-metal-to-phosphorus ratio is used in the method. In some embodiments, the algorithm or model may be trained with artificial intelligence (for example, machine learning) to take other sediment metrics into account to determine what conditions preferentially lead to the efficient formation of alkaline-insoluble, calcium phosphate minerals.

Continuing to refer to FIG. 2, in one embodiment the second general step 200 of FIG. 1 (calculation of one or more coefficients and/or indices for one or more water body and sediment quantifiable properties) includes a first substep 202, a second substep 204, and a third substep 206. In one embodiment, in the first substep 202 (or a fourth overall step) at least one coefficient of a group of coefficients relating to sediment characteristics (referred to herein as “sediment characteristic coefficients”) is calculated using the values determined in the first substep 102 (water body characteristics initial input data), the second substep 104 (sediment characteristics initial input data), and the third substep 106 (sediment extraction initial input data/sediment characteristics initial input data) of the first general step 100 (for example, as shown in FIG. 3). In some embodiments, the sediment characteristic coefficients that may be calculated in this substep include, but are not limited to:
a. Sediment expansion coefficient;
b. Sediment disturbance coefficient;
c. Iron stripping coefficient.

Continuing to refer to FIG. 2, in one embodiment in the second substep 204 (or a fifth overall step), at least one index of a group of indices relating to water column release characteristics (referred to herein as “water column release initial indices”) is calculated using the values determined in the first substep 102 (water body characteristics initial input data), the second substep 104 (sediment characteristics initial input data), and the third substep 106 (sediment extraction initial input data/sediment characteristics initial input data) of the first general step 100, and the sediment characteristic coefficients determined in the first substep 202 of the second general step 200 (for example, as shown in FIG. 3). In some embodiments, the water column release indices that may be calculated in this substep include, but are not limited to:
a. Oxygen depletion index;
b. Diffusion index;
c. Redox release index;
d. Organic phosphorus release index.

Continuing to refer to FIG. 2, in one embodiment in the third substep 206 (or a sixth overall step), a total water column release index is determined using the water column release initial indices calculated in the second substep 204 of the second general step 200, and then used to estimate the phosphorus release from the sediment sample over time. In some embodiments, the phosphorus release from the sediment sample is estimated over a period of day(s), week(s), month(s), quarter(s), year(s), decade(s), and/or in total (over an unlimited time frame). However, it will be understood that phosphorus release over different or various spans of time may be used.

Continuing to refer to FIG. 2, in one embodiment the third and fourth general steps 300, 400, and the fifth and sixth optional steps 500, 600, of FIG. 1 do not include further substeps, although it will be understood that in some embodiments one or more substeps may be included in one or both general steps. These steps are shown accordingly in FIG. 2, and the discussion above of these steps in FIG. 1 is applicable to FIG. 2.

Continuing to refer to FIG. 2, in a third step 300 (or a seventh overall step), a predicted total phosphorus release value is calculated. In one embodiment, the predicted total phosphorus release value is a predicted total phosphorus release value over time. In one embodiment, the predicted total phosphorus release value is calculated using at least the total water column release index calculated in the third substep 206 of the second general step 200. In some embodiments, additional values, coefficients, and/or indices may also be used. In some embodiments, the predicted total phosphorus release value is calculated using the predicted aluminum binding efficiency value calculated in the fourth substep 208 of the second general step 200 in addition to the total water column release index and any additional values, coefficients, and/or indices.

Continuing to refer to FIG. 2, in the fourth step 400 (or an eighth overall step), a refined predicted total phosphorus release value is generated. In some embodiments, the refined predicted total phosphorus release value represents more accurate, measured phosphorus release over time value is determined from laboratory incubations, mesocosm experiments, limnocorrals, and/or real-world bottom water phosphorus sampling and used to provide feedback data to the model which incorporates the various data sources to calculate coefficients and build the water column release index. In one embodiment, the refined predicted total phosphorus release value is generated by inputting feedback data and/or additional or updated water body and sediment characteristics initial input data (collectively, referred to herein as feedback input data). In some embodiments, the coefficients and/or indices calculated in the first substep 202, and second substep 204, and/or the third substep 206 of the second general step 200 are updated or modified based in the entry of the feedback input data. Feedback input data may be entered in real time as it is obtained (manually, automatically, and/or semi-automatically), may be entered at a fixed schedule (for example, hourly, daily, weekly, or monthly, or at pre-set intervals), may be entered randomly, and/or at other pre-determined or unknown times or time intervals. Thus, this step establishes a feedback loop within the system that refines predictions and accuracy (for example, as shown in FIG. 4). In some embodiments, the feedback input data can be further improved by measuring change in sediment characteristics initial input data over time, such as in laboratory incubations, mesocosm experiments, limnocorrals, and/or real-world water body sediments. In some embodiments, the feedback input data can be further improved by using artificial intelligence processes such as machine learning to improve the model based on changes in the sediment phosphorus fractionation after a phosphorus binding treatment is applied to the sediment of the water body and sediment subsequently sampled.

Continuing to refer to FIG. 2, in one embodiment in the optional fifth step 500 (or a ninth overall step), the efficacy or efficiency of a suggested method of treatment of the water body is predicted (referred to herein as “treatment efficacy prediction”). In one embodiment, this prediction is based on the water body and/or sediment characteristics initial input data (for example, from the first step 100 of the method) and/or the calculated coefficients and/or indices (for example, from the second step 200) of the method. This treatment efficacy prediction may be used to evaluate one or more proposed treatments to determine which one(s) would be the most effective in binding and/or removing phosphorus from the water body, based on the properties of that water body and its sediment. Additionally or alternatively, the treatment efficacy prediction may be used as a mitigating or exacerbating factor in the determination of the predicted total phosphorus release value and/or the refined predicted total phosphorus release value.

Continuing to refer to the optional fifth step 500 shown in FIG. 2, in one embodiment, one suggested method of treatment is the application of aluminum (for example, the application of aluminum salts) to the water body to bind phosphorus. In one embodiment, the aluminum salts include, but are not limited to, aluminum sulfate and/or aluminum chlorohydrate. In one embodiment, the predicted aluminum binding efficiency value is estimated based on an aluminum-to-phosphorus ratio in the metal-oxide fraction (sediment extraction characteristics initial input data, as calculated in the third substep 106 of the first general step 100), the sediment pH (sediment characteristics initial input data, as determined in the second substep 104 of the first general step 100), the metal-oxide phosphorus concentration of the natural sediment (sediment extraction characteristics initial input data, as calculated in the third substep 106 of the first general step 100), the depth in the water column at which the sediment sample was collected below the surface of the water body (water body characteristics initial input data, as calculated in the first substep 102 of the first general step 100), and annual pH range of the bottom water of the water body (water body characteristics initial input data, as calculated in the first substep 102 of the first general step 100). The predicted aluminum binding efficiency value indicates an efficiency of aluminum to suppress phosphorus release, which affects the overall result within the water body of the predicted total phosphorus release. For example, if the predicted aluminum binding efficiency value is high, a lower dose of an aluminum-based phosphorus binding compound may be used to suppress the release of sediment phosphorus in order to manage water quality. In some embodiments, the aluminum efficiency value is not used in the calculation of the predicted total phosphorus release in the third step 300 and/or the fourth step 400, but is used to adjust a predicted effect of the predicted total phosphorus release on the water body being evaluated (for example, as a mitigating or exacerbating factor). On the other hand, in some embodiments the aluminum efficiency value is used in the calculation of the predicted total phosphorus release if it is anticipated that aluminum phosphorus could become releasable in the future, such as lake or reservoir drawdown which decreases the depth of the water column and exposes the sediment aluminum-bound phosphorus to the epilimnion and a higher pH or the excessive input of nutrients that could stimulate eutrophication and a high pH.

Referring now to FIG. 3, a flow diagram of a method in accordance with the present disclosure is shown. In particular, the method shown in FIG. 1 illustrates the relationships between measured parameters (site information, sediment sequential extraction data, and sediment properties), calculated indices and coefficients, a water column release index, a predicted aluminum binding efficiency, and a predicted total phosphorus release over time. In one embodiment, the measured parameters are used to calculate one or more coefficients and indices, which are used to generate a predictive result. In one embodiment, as shown in FIG. 1, the predictive result is a predicted total phosphorus release value.

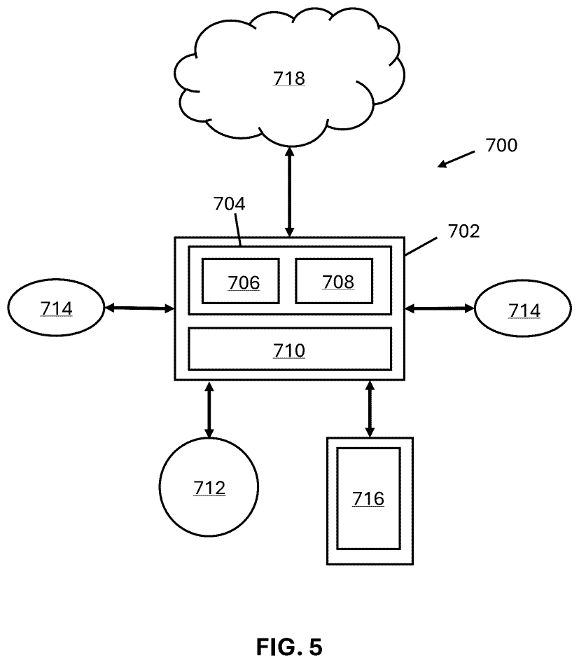

Referring now to FIG. 5, an exemplary system for performing the method is shown. In one embodiment, the system 700 generally includes a computer 702 having processing circuitry 704 including a memory 706 and a processor 708. In one embodiment, the memory 706 is in communication with the processor 708 and has instructions that, when executed by the processor 708, configure the processor 708 to perform algorithm, calculations, modeling, predictions, and/or the like according to the method discussed herein. That is, in one embodiment, the processing circuitry 704 is programmed or programmable to perform and entirety of or at least a portion of the method discussed herein. In addition to a traditional processor 708 and memory 706, the processing circuitry 704 may include integrated circuitry for processing and/or control, such as one or more processors and/or processor cores and/or FPGAs (Field Programmable Gate Array) and/or ASICs (Application Specific Integrated Circuitry). The processing circuitry 704 may include and/or be connected to and/or be configured for accessing (for example, writing to and/or reading from) the memory 706, which may include any kind of volatile and/or non-volatile memory, such as cache and/or buffer memory and/or RAM (Random Access Memory) and/or ROM (Read-Only Memory) and/or optical memory and/or EPROM (Erasable Programmable Read-Only Memory). Such memory 706 may be configured to store code executable by control circuitry and/or other data, such as data pertaining to communication. Further, the processing circuitry 704 may be configured to control any of the methods described herein and/or to cause such methods to be performed, such as by the processor 708. Corresponding instructions may be stored in the memory 706, which may be readable and/or readably connected to the processing circuitry 704. In other words, processing circuitry 704 may include a controller, which may comprise a microprocessor and/or microcontroller and/or FPGA (Field-Programmable Gate Array) device and/or ASIC (Application Specific Integrated Circuit) device. In some embodiments, the processing circuitry 704 includes or may be connected or connectable to the memory 706, which may be configured to be accessible for reading and/or writing by the controller and/or processing circuitry 704. In some embodiments, the computer 702 further includes one or more additional modules, such as a communications module 710 that is configured to send and receive data from one or more remote devices, sensors, control units, computers, remote storage devices, networks, and the like, and to transmit such data to and from the processing circuitry 704. In some embodiments, the water body and sediment characteristics initial input data, calculated coefficients and indices, predicted total phosphorus release value, feedback input data, and/or other data are stored in a database in the memory 706 and/or other data storage device, from where it can be accessed by the processor 708 and/or artificial intelligence system, and is available for access and use in the predictive model. In some embodiments, each of the types of initial input data, coefficients, indices, feedback input data, and/or other data is stored in a respective database. Thus, in some embodiments, the method is a computer-implemented method for predicting total phosphorus release from a sediment within a water body and/or a method for recommending a method of treatment of the water body (for example, a method or system of binding and/or removing phosphorus and/or other nutrients, and/or a method of controlling aquatic algae and/or plants that release nutrients) and/or a method for predicting the efficacy of a particular method of treatment of the water body.

Continuing to refer to FIG. 5, in some embodiments, the computer 702 includes, is in communication with, or is configured to be in communication with an artificial intelligence system 712. In one embodiment, the artificial intelligence system 712 is in communication with the processing circuitry 704 such that the artificial intelligence system 712 and the processing circuitry 704 together perform the activities (for example, determinations, calculations, predictions, comparisons, and the like) discussed herein. As used herein, the term “artificial intelligence” refers to smart machines and/or processors capable of performing tasks that typically require human intelligence and that are capable of learning from experience, adjusting to new inputs, processing large amounts of data, and recognizing patterns in the data, and, in some embodiments, doing so by autonomous or semi-autonomous action. As used herein, this term also includes machine learning. In one embodiment, the artificial intelligence system 712 is configured to collect or receive initial input data and/or feedback input data, to use that data to generate the indices and/or coefficients, perform additional calculations and data manipulations as needed, make correlations between the data, indices, and/or coefficients, make predictions based on the correlations, determinations, and/or correlations, make adjustments of predictions, correlations, determinations, and/or correlations based on feedback input data and/or other data or inputs, recognize patterns, and/or perform other tasks relevant to execution of the method discussed herein. In one embodiment, the artificial intelligence system predicts the total phosphorus release and the refined predicted total phosphorus release value. In some embodiments, the artificial intelligence system 712 predicts the aluminum binding efficiency and/or evaluates various treatment options for the water body to determine their efficacy based on the data obtained from the water body.

Continuing to refer to FIG. 5, in some embodiments, the system may further include one or more sensors or sensing devices 714 that are configured to measure one or more items of initial input data and/or feedback input data. In one embodiment, the computer 702 is in wired and/or wireless communication with the sensing devices 714, directly and/or indirectly through one or more intermediate devices or computers (for example, data input devices, tablet computers, handheld computers, or the like), such as peripheral device(s) 716. In one embodiment, the communications module 710 is configured to send data to and receive data from the one or more sensing devices 714. Thus, in some embodiments, the methods discussed herein include transmitting data to the processing circuitry 704 from sensing device(s) 714, peripheral device(s) 716, and/or from other sources, and/or receiving data from the processing circuitry 704 via sensing device(s) 714, peripheral device(s) 716, and/or other devices (for example, through the communications module 710). In some embodiments, the one or more sensing devices 714 include, but are not limited to, pH sensors, scales, microscopes, spectroscopes, and other data-gathering equipment suitable for obtaining the initial input data and/or feedback input data. In some embodiments, the system further includes one or more peripheral devices 716. For example, the peripheral devices 716 may include one or more devices configured to send and/or receive data, such as data entered by a user, one or more remote data storage devices, one or more computers, tablets, phones, control units, or the like. Additionally, in some embodiments, the system 700 is in communication with, or is configured to be in communication with, one or more networks, such as a cloud network 718.

Referring again to the method discussed herein, the following examples illustrate the performance and/or use of selected method steps.

### EXAMPLE 1

In a first non-limiting example (Example 1), a method of determining the sediment expansion coefficient is described. In one embodiment, the sediment expansion coefficient, a metric to quantify the ability of a sediment sample to expand at the sediment-water interface, is determined using a simple analytical procedure. In one embodiment, the propensity of the surficial sediment to expand at the sediment-water interface and to release contaminants into the overlaying water column is determined using a calculations and algorithms from analytical laboratory measurements. This determined value is input into a model which can be refined with additional laboratory measurements of other sediment samples, including, but not limited to, their analytical characteristics, their behavior in water, in laboratory experiments, and in the real world. In one aspect of the embodiment, a sediment expansion coefficient is determined using a calculation of the expanded dry bulk density and the compacted dry bulk density to quantify how much the sediment expands in the presence of water and in the absence of compaction pressure (at the sediment-water interface) and how much can be compacted under the weight of additional sediment. The sediment expansion index is a metric which can quantify the amount or extent of muck within a specific area of the water body. The understanding of muck accumulation using the sediment expansion coefficient within a lake can also provide insightful information about the biogeochemical cycling within the sediment of that region of the lake, especially when combined with other parameters, physical properties, and biogeochemical analyses.

In one embodiment, the sediment expansion coefficient is determined by first measuring the expanded dry bulk density and the compacted dry bulk density. Next, the compacted dry bulk density is divided by the expanded dry bulk density, according to Equation 1 below. This coefficient describes the amount of expansion that occurs when the sediment is placed in water at a low pressure, with a value of two demonstrating that the dry bulk density is twice a high when the sediment is compacted.

$$Equation 1$$

$$Sediment Expansion Coefficient ( unitless ) = \frac{Compacted dry bulk density ( \frac{g}{{cm}^{3}} )}{Expanded dry bulk density ( \frac{g}{{cm}^{3}} )} = \frac{\frac{mass of dry particles ( g )}{volume of dry particles ( {cm}^{3} )}}{Wet bulk density ( \frac{g}{{cm}^{3}} ) \times \% solids}$$

Two alternative methods to this procedure can be utilized to qualitatively determine the sediment samples propensity to expand in the absence of pressure and compress under pressure. The first method involves the measurement of the dry bulk density with a coring device, where a coring device to obtain a core of the sediment sample and transporting back to the laboratory. At the laboratory, the core can be cut so that a known volume of sediment is obtained from a specific section within the core, using the length of the section of the core and diameter of the cylinder. Then, the sediment of a known volume can be dried and weighed, with the dry bulk density being calculated as the mass of dried sediment divided by the known volume of the wet sample. The dry bulk density is then measured for various depth layers (for example, from 0-3 cm, from 3-6 cm, 6-9 cm, etc.), down to a depth of at least 20 cm. The deepest layer of the sediment profile is the most compacted and will therefore demonstrate the highest dry bulk density. The sediment expansion coefficient is then calculated by dividing the dry bulk density of the deepest layer by the dry bulk density of each individual layer above that sample. This procedure benefits from the ability to compare the sediment expansion coefficient with depth throughout the profile of the sediment, allowing for the determination of the approximate depth where expansion becomes more significant and the approximate depth where compaction occurs. The second method involves placing a known volume of wet sediment into a centrifuge container, preferably 50 mL or 250 mL and centrifuging at 3,000-5,000 RPM for 5-10 minutes to cause the sediment particles to compact. The change in volume in the volume of the sediment after centrifugation can also be used as the sediment expansion coefficient. For example, a change from 50 mL to 25 mL entails a sediment expansion coefficient of 2.

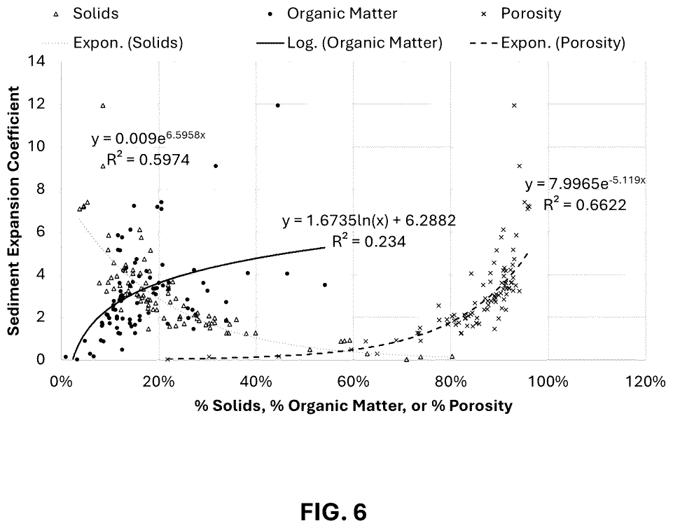

In an exemplary experiment in Example 1, the sediment expansion coefficient was measured for 90 sediment samples taken from various depths in 18 different waterbodies. Organic matter, porosity, and were also performed on all 90 sediment samples to look for correlations with the sediment expansion coefficient. Percent solids was measured as described above. The organic matter content, percent solids, and as the mass of dry sediment divided by the change in water volume due to displacement. The relationship between the sediment expansion coefficient and the percent solids, the percent organic matter, and porosity is plotted in FIG. 6. FIG. 6 shows a strong positive relationship between the sediment expansion coefficient and percent solids (exponential, R²=0.5974) and the sediment expansion coefficient and porosity (exponential, R²=0.6622). There is also a moderate correlation between the sediment expansion coefficient and organic matter (logarithmic, R²=0.234). It is reasonable that the organic matter correlation is less pronounced, likely due to the difference in the behavior of organic-rich sediments with large particles (diminishing increase in the sediment expansion coefficient with increased organic matter content) and organic-rich sediments with fine particles (continuing linear increase in the sediment expansion coefficient with increased organic matter content).

The analysis of 90 lake sediment samples revealed that the minimum sediment expansion coefficient was 0.03, the twenty fifth percentile was 1.9, the median was 2.8, the average was 3.1, the seventy fifth percentile was 3.7, and the maximum was 11.9. This distribution is useful for qualitatively characterizing the amount of muck in new sediment samples. The depth of releasable phosphorus is commonly estimated as 10 cm when sediment phosphorus management is considered and the entire mass of releasable phosphorus needs to be calculated. This model compares the sediment expansion coefficient of a sediment sample within the database of previously analyzed sediment samples. This allows for a better estimation of how expansive the lake sediment is and how deep contaminants such as phosphorus can be released from compared to other lakes that have similar water quality, trophic statuses, bathymetries, weather patterns, watersheds, or other biogeochemical parameters. This enhanced understanding may help to explain why some sediments with lower concentrations of releasable phosphorus experience enhanced water quality degradation compared to other lakes that have similar watersheds, weathers, food webs, and depth profiles.

The sediment expansion coefficient can vary significantly within a particular lake or it may be relatively consistent, dependent on a variety of biogeochemical factors at play. In one large lake (>100,000 acres) with a complex bathymetry and multiple basins, referred herein throughout as Lake 1, 15 sediment samples were collected and analyzed. The sediment expansion coefficient in Lake 1 sediment samples ranged from 1.6 to 4.3, with an average value of 2.9, a standard error of 0.19, and no correlation between depth and the sediment expansion coefficient, indicating no particular pattern of muck (low density sediment) distribution within the lake based on depth. In another smaller lake (˜800 acres) with a simple bowl like bathymetry, herein referred to as Lake 2, three samples were collected from a shallow, mid-depth, and deep zone in the lake. The sediment expansion coefficient in Lake 2 sediment samples ranged from 0.9 to 11.9, with an average value of 5.5, a standard error of 3.3, with a low sediment expansion in the shallow zone, a high value in the mid depth site, and a medium value in the deep site, indicating an accumulation of muck in the mid depth zone of the lake. In a third lake, with a moderate size (˜1,400 acres) and moderate bathymetric complexity, herein referred to as Lake 3, three samples were collected, the sediment expansion coefficient ranged from 0.5 to 4.6, with an average value of 2.56, a standard error of 1.2, and a substantially higher sediment expansion coefficient in the deep zone of the lake, indicating an accumulation of muck in the deepest zone of the lake.

The sediment expansion coefficient by itself has no meaning or intrinsic value for the vast majority of sediments, which will demonstrate at least some degree of expansion. However, this analysis can be used to compare the sediment expansion coefficient between sediment samples within a particular lake and within a database, with the ultimate goal of better determining the depth of sediment in which a given contaminant will be released both over time and in total. The depth of releasable phosphorus is commonly estimated as 10 cm when sediment phosphorus management is considered and the entire mass of releasable phosphorus needs to be calculated. However, as this data set shows, some sediment within a particular lake may expand much more readily than sediment in a different part of the lake and using the 10 cm depth of releasable phosphorus for all sites will lead to the overestimation of the amount of phosphorus that could be released in sediments that are more compact and an underestimate in sediments that expand more easily. Using the sediment expansion coefficient can allow for a more even dose of a phosphorus binding compound which will ultimately provide a large benefit in water quality at the exact same price as a dose where the depth of sediment is assumed to be 10 cm throughout the lake.

### EXAMPLE 2

In a second non-limiting example (Example 2), an index that quantifies the rate of oxygen depletion at the sediment-water interface of a site within a water body, as well as the extent, duration, and timing of anoxia at sites within a water body is determined using site characteristics. In one embodiment, the sediment expansion coefficient is utilized with the percent dissolved manganese, the labile organic matter content of the sediment and the site-specific Osgood index to provide an enhanced understanding of oxygen depletion and the potential for anoxia at each site within the lake.

In a first experiment in Example 2, a model used the site-specific Osgood index to estimate the rate of oxygen replenishment to the sediment, the labile organic matter content and the sediment expansion coefficient to estimate the rate of oxygen depletion within the sediment, and the percent dissolved manganese to adjust the model, resulting in the creation of an oxygen depletion index that could accurately determine the potential for each site where a sediment sample was collected to experience anoxia and the extent of anoxia at the site.

In one embodiment, the site-specific Osgood index is a slight modification of a classic lake management parameter, the Osgood index. The Osgood Index is essentially a ratio of the average depth (m) to the surface area (square root of km²) that provides a general understanding of the lake's ability to resist mixing, with a larger Osgood index being more likely to resist mixing and maintain thermal stratification through a season. Deeper lakes have a greater ability to form a significant temperature gradient between the surface and bottom water, which requires a stronger physical force to mix the layers. Lakes with larger surface areas experience greater shear induced mixing due to a greater transfer of energy from wind stress compared to lakes of the same depth but smaller surface areas. Therefore, a shallower lake with a small surface area would have a similar potential to stratify compared to a deeper lake with a larger surface area if the Osgood index is the same.

Generally, a lake with Osgood index of 6 or greater indicates the lake is more protected and more likely to remain stratified during the summer, whereas lakes with an Osgood index of less than 6 are considered more likely to mix. Ultimately, weather conditions. geographic location, and bathymetry also factor into the potential to mix, but the Osgood index is a generally applicable metric. Although the Osgood index is helpful for understanding mixing within an entire lake, it is does not provide information about the mixing at an particular site within the lake, which is why using the depth of the specific site, rather than the average depth of the lake, may be more helpful for the model.

The potential for a specific site to mix or remain stratified was based on the same metric of a site specific Osgood index indicating a propensity to remain stratified and a site specific Osgood index of less than 6 indicating a potential to mix during the summer. The potential for a specific site to mix or remain stratified provides useful information about the replenishment of oxygen, which can be depleted in stratified lakes with organic rich sediment. Therefore, locations where the site-specific Osgood index is less than 6 are more likely to have continual oxygen replenishment, while sites where the site specific Osgood index is greater than 6 are less likely to have oxygen delivered to the sediment during the summer due to a stronger potential to stratify.

The rate of oxygen depletion is an important consideration for the prediction of anoxia, as strongly stratified lakes (for example, Lake Tahoe) can retain adequate dissolved oxygen at the sediment-water interface due to a very low oxygen demand, even when no new oxygen is delivered to the bottom water for months or years. The sediment oxygen demand can be measured in a lab, but this measurement is complex, expensive, and time consuming. The present model provides an estimation of sediment oxygen demand using labile organic matter content and the sediment expansion coefficient. Using labile organic matter (loss on ignition at 250° C.) instead of total organic matter (loss on ignition at 500° C.) avoids the inclusion of non-mineralizable organic compounds such as humic and fulvic acids, which can be a substantial portion of the total organic matter in aquatic sediments and overexaggerate the truly mineralizable organic matter content of the sediment. The sediment expansion coefficient and porosity are critical for an optimized understanding of the extent of the oxygen depletion due to the mineralization of organic matter in the sediment, as more compacted sediment does not allow for the rapid diffusion of dissolved organic matter below the sediment-water interface and the depth of accessible organic matter by aerobic organisms is minimal. Expansive sediment with a high porosity provides the path for rapid penetration of dissolved oxygen beneath the sediment-water interface and substantially increases the depth of accessible organic matter by aerobic organisms.

The percent dissolved manganese in the sediment is used to calibrate the model, as it serves as a conservative tracer of anoxic conditions. Manganese oxides are only readily reduced when oxygen is depleted and the dissolved manganese that is produced as a biproduct of manganese reduction is not oxidized very rapidly, generally persisting for months, unlike iron, which is rapidly oxidized. Therefore, a high concentration of dissolved manganese is an indicator that the sediment has been anoxic if only the surface sediment is sampled. However, the understanding of how to relate the percent dissolved manganese is complex, as it is highly seasonally dependent. The most appropriate method to utilize the percent dissolved manganese in the surficial sediment sample is to reference a database which correlates dissolved manganese with relevant parameters, such as seasonality, site specific Osgood index, sediment expansion coefficient, labile organic matter content, sediment oxygen demand, as well as extent, duration, and timing of anoxia.

A simpler approach is to measure the dissolved manganese of sediment samples where the extent, duration, and timing of anoxia is known and use these as points of comparison to other samples from the same lake, where the extent, duration, and timing of anoxia is unknown. Then, the sediment expansion coefficient, the labile organic matter content, and the site specific Osgood index can be used to inform the model about the biogeochemical processes that are affecting the development or lack of anoxia at each site. In this non-limiting example, data inferred from sediment samples that were collected in December from the smaller and simple lake, Lake 2, which was described in Example 1, were used in the model. Lake 2 had mixed roughly two months prior to the collection of the sediment samples. The sediment sample from a deep, strongly stratified zone in a lake where anoxia is extensive in the summer displayed 33% dissolved manganese. The shallow site where the water column is permanently mixed and the sediment-water interface is permanently oxic displayed 23% dissolved manganese. A sediment sample from the same lake at a moderate depth and an unknown mixing and anoxic regime displayed 38% dissolved manganese, indicating that anoxia was even more intense at the mid-depth site in this lake. A qualitative sediment-oxygen demand study revealed that the sediment from the mixed depth lake became anoxic more than twice as quickly as the deep lake sediment when held under identical conditions.

In Lake 2, the mid-depth sediment sample displayed the largest sediment expansion coefficient (11.9) and the largest labile organic matter content (26%), in addition to the greatest percent dissolved manganese, while the deep site that was suspected to be the most anoxic only had a sediment expansion coefficient of 3.6, a labile organic matter content of 9%, and a dissolved manganese of 33%. The site specific Osgood index of the deep site (15.3) showed that it was permanently stratified, while the mid-depth site had a site specific Osgood index of 6.3, which is on the edge of being fully stratified throughout the summer. These biogeochemical insights allowed the model to characterize the mid-depth site as the “goldilocks zone,” where anoxia would quickly develop in the summertime and partial mixing would redeliver oxygen to the sediment while mixing phosphorus rich bottom-water into the photic zone and lead to water quality degradation and the accumulation of more labile organic matter after algae blooms died and deposited biomass back in the sediment and the replenishment of electron acceptors such as oxygen, nitrate, and iron that were reintroduced after partial mixing events. This constant cycling back and forth between oxic and anoxic conditions led to much more pronounced anoxia than the deep zone, which stayed permanently stratified, but did not received nearly as much labile organic matter or new electron acceptors during the summer. This complex biogeochemical cycle also allowed for the accumulation of higher concentrations of phosphorus, redox sensitive iron, and total organic matter in the mid-depth “goldilocks zone,” which commonly occurs in the deep zone of other lakes. The model was then able to utilize this complex information to better estimate the releasability of the releasable fractions of phosphorus in the lake.

### EXAMPLE 3

In a third non-limiting example (Example 3), the releasability of the organic phosphorus fraction in a lake sediment sample is estimated using the depth of the water column, the carbon to phosphorus ratio, the percentage of the total organic matter that is labile (% labile organic matter) and the oxygen depletion index. In one embodiment, the organic phosphorus releasability is determined by a model which incorporates a variety of biogeochemical parameters to better inform the required dose of a sediment phosphorus binder. In one aspect of the embodiment, the model determines the organic phosphorus releasability to provide a more accurate estimate of the total mass of releasable phosphorus in order to provide a report for a customer who paid for an analytical laboratory analysis of sediment phosphorus forms. In one aspect of the embodiment, the enhanced understanding of the releasability of organic phosphorus is used to determine the best time frame to apply a sediment phosphorus binding compound.

In a first experiment in Example 3, the utilization of the biogeochemical parameters in this analysis to elucidate the releasability of organic phosphorus extracted from a sediment sample ultimately leads to a more accurate calculation of the mass of releasable phosphorus and the prioritization of the sediment for the management of sediment phosphorus. Although many organic phosphorus forms that are extracted by 0.1 M NaOH are bioavailable, some forms are not. The non-bioavailable form are generally contained within large organic structures like lignin, lignocellulose, humic acid, and recalcitrant organic compounds that have been partially degraded. The depth of the water column, the percentage of organic matter that is labile or lost at 250 C, and the carbon to phosphorus ratio of the sediment are determined for a sediment sample and incorporated into a model that predicts the overall releasability (as a percentage) of the organic phosphorus that was extracted from the sediment sample.

The depth of the water column has a twofold influence in the releasability of the organic phosphorus. The first factor is that a shallower depth will experience warmer temperatures during the summer, which correspond with enhanced respiration and increased microbial degradation of organic compounds. Many organic phosphorus compounds contain highly energetic phosphoanhydride bonds which can store energy that can be released when microorganisms use enzymes to break them. An example is adenosine triphosphate (ATP), which is one of the most common organic phosphate molecules and is widely used to store cellular energy. ATP is converted into adenosine diphosphate (ADP) and inorganic phosphate by microorganisms that require energy for metabolism. Therefore, many organic phosphorus compounds are broken down and inorganic phosphate is released during microbial respiration, which increases in magnitude as the sediment warms. The second aspect where the depth of the water column is an important consideration for the model is due to the degradation of highly labile organic matter and the associated bioavailable organic phosphorus while the organic matter slowly settles through the water column. The settling process can take weeks in deeper water bodies, where the organic matter that eventually reaches the sediment has already lost the vast majority of bioavailable phosphorus. Alternatively, highly bioavailable organic matter containing highly bioavailable phosphorus can be deposited to the sediment shortly after an aquatic photosynthetic organism dies in a shallow water body.

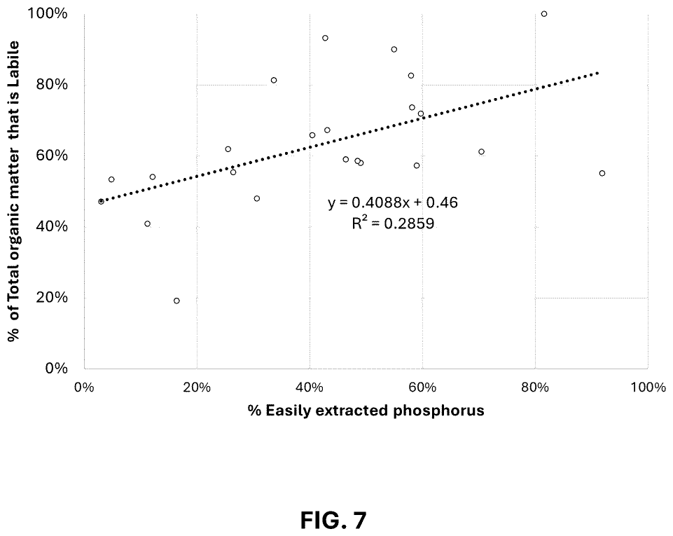

The bioavailability of the organic matter within a sediment sample can be qualitatively understood by diving the labile organic matter content by the total organic matter content to determine the percentage of phosphorus that is highly bioavailable. Highly degraded organic matter is rich in humic acid and fulvic acid and these compounds are stable up to 250° C., so they will not be included in the labile organic matter content, but will be determined in the total organic matter content. An extraction of labile phosphorus and an analysis of the percent labile organic matter was performed on 23 different aquatic plants and algae. This analysis involved the high speed blending of the fresh organic matter to break and lyse cells, followed by the extraction of the highly releasable organic phosphate using 1 M ammonium chloride, which expels intracellular phosphate and organic phosphorus through ionic displacement and increased permeability of cell membranes due to conductivity gradients. The total phosphorus content, the labile organic matter and total organic matter of homogenized subsamples of each plant or algae sample was also analyzed. The results are shown in FIG. 7 and demonstrate how an increase in the percentage of labile organic matter (out of total organic matter content) of aquatic plants and algae can be indicative of a greater concentration of highly releasable organic phosphorus within the organic matter. Some of these plants were also left out at room temperature and covered in water, to simulate the natural process of degradation in aquatic environments. Afterwards, the remaining material was washed and reanalyzed. All plants demonstrated a reduction in the labile organic matter content (an increase in non-labile organic matter by weight) and a reduction in easily extractable organic phosphorus, as the highly releasable organic phosphorus was released into the water and only the non-releasable portion remained.

The carbon to phosphorus ratio, which can be calculated using the molar ratios of the organic matter content (assumed to be 58% carbon by mass) and the organic phosphorus content, is also a good indicator of the extent of degradation of the organic matter. Previous research has shown that the degradation of organic matter leads to a more rapid release of phosphorus compared to the loss of carbon, resulting in an increase in the carbon to phosphorus ratio. A general starting point of the Redfield ratio or 106 carbon to 1 phosphorus can be assumed and used in the model. A carbon-to-phosphorus ratio that is significantly higher than the Redfield ratio (>400 to 1) can indicate highly degraded organic matter with a very low percent of releasable organic phosphorus. Alternatively, a very low carbon-to-phosphorus ratio generally indicates high bioavailable organic matter and highly releasable organic phosphorus. Polyphosphates are highly bioavailable phosphorus compounds that extracted in the organic phosphorus fraction actually has no associated carbon atoms therefore its presence decreases the carbon to phosphorus ratio. Polyphosphates are produced by aerobic organisms in the presence of oxygen during the microbial respiration and are commonly observed in wastewater treatment sludge. Although polyphosphates accumulate during oxic conditions, they are utilized by anaerobic bacteria as a source of energy in the absence of oxygen and converted into inorganic phosphate, which is released from anoxic sediment. Sediment samples that are in the “goldilocks zone” often exhibit a carbon to phosphorus ratio below 106 in the winter, indicating the presence of high concentrations of polyphosphates that are produced by the aerobic organisms that colonize the sediment during the winter and metabolize the sediment organic matter that accumulated during the summer after the death and settling of algae. Therefore, a low carbon to phosphorus ratio can also indicate the potential for a high release of organic phosphorus in sediments that have a site specific Osgood index around 6, organic matter and organic phosphorus rich sediment.

### EXAMPLE 4

In a fourth non-limiting example (Example 4), the binding efficiency of aluminum salts to suppress phosphorus release is determined by using the natural aluminum to phosphorus ratio of the metal oxide phosphorus fraction and incorporating the depth and sediment pH to understand how the predicted aluminum binding efficiency changes within different locations of the water body. In one embodiment, the aluminum salt is aluminum sulfate. In another aspect of the embodiment, the aluminum salt is aluminum chlorohydrate.

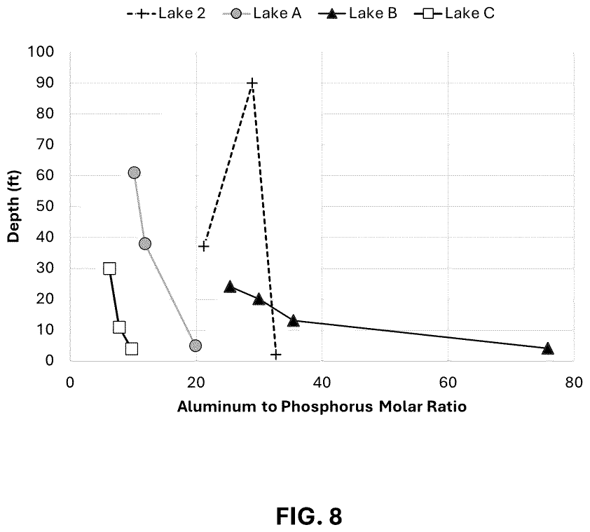

In a first example, the depth of various sediment samples from within different lakes is determined and sediment samples are collected at a shallow, medium depth, and deep depth location in each lake. Lake 2, which was described in Example 1 and Example 3 and three additional lakes; Lake A, Lake B, and Lake C, where also included in this analysis. A sequential phosphorus extraction of all sediment samples was performed and the aluminum concentration of the metal oxide fraction (0.1 M NaOH) was determined with an ICP-OES, along with the metal-oxide or aluminum associated phosphorus which was determined using spectrophotometry and the molybdenum blue method, EPA 365.3. The results in FIG. 8 demonstrate how the aluminum to phosphorus ratio is highly variable amongst different lakes and even within a single lake, at locations with different depths. Lake A, B, and C were highly productive, hypereutrophic lakes, while Lake B is mesotrophic or low level eutrophic. These data are aligned with the understanding that shallow, productive sediments can be susceptible to aluminum phosphorus release due to the high pH that can desorb aluminum bound phosphorus through the formation of Al(OH)₄⁻ that will release any phosphate that may have been bound. However, interestingly enough, even though Lake C demonstrated a lower aluminum-to-phosphorus ratio in the shallower sediment, it was still demonstrated a very high phosphorus binding efficiency which would challenge the simple notion that aluminum is a poor phosphorus binder in shallow, productive lake sediments. An alternative explanation may be that the aluminum is remaining reactive through interactions with high pH water at the sediment-water interface and when it is buried beneath the sediment-water interface and protected from the high pH, it is very efficient at binding the inorganic phosphorus that is present. In Lake B, a lower phosphorus binding efficiency of aluminum was observed in shallow sediments that exhibited a low pH. The low pH demonstrates is due to the respiration that occurred during the transportation and processing of the sediment, which was highly organic in nature. This low pH in the laboratory demonstrates a low buffering capacity and the likely potential that the sediment-water interface would experience a very high pH in the real world during the periodic and extensive algae blooms that frequently occur in the lake. Ultimately, these data demonstrate biogeochemical and physical parameters can provide insight that can explain some, but not all of the factors behind complex interactions such as the efficiency of aluminum in binding phosphorus in lake sediments. The model that incorporated this data utilizes new data input to continually refine predictions and has become increasingly predictive of the aluminum to phosphorus binding ratio of lake sediments before the information is provided to the modeling program. A model that has the ability to refine itself can be used to estimate more complex geochemical processes such as the ability of calcium to bind phosphorus, as many different forms of calcium phosphate minerals with varying solubilities exist based on pH, alkalinity, organic matter.

TABLE 1
Data associated with the aluminum phosphorus binding efficiency of
lake sediments analyzed and incorporated into the predictive model.

| | | | | | | Metal Oxide |
|---|---|---|---|---|---|---|
| | Trophic | Site | Depth | Al to P | pH of | Al Content |
| Lake | Status | Depth | (ft) | Ratio | Sediment | (mg/kg) |
| 2 | Mesotrophic/ | Shallow | 2 | 32.7 | 6.81 | 507 |
| | Low level | Medium | 37 | 21.3 | 6.81 | 989 |
| | Eutrophic | Deep | 90 | 28.9 | 6.89 | 1,119 |
| A | Hypereutrophic | Shallow | 5 | 19.9 | 6.98 | 99 |
| | | Medium | 38 | 11.9 | 6.88 | 295 |
| | | Deep | 61 | 10.1 | 6.88 | 381 |
| B | Hypereutrophic | Shallow | 4 | 75.8 | 5.55 | 3,090 |
| | | Medium | 13 | 35.4 | 6.09 | 3,689 |
| | | Medium | 20 | 29.9 | 5.60 | 3,377 |
| | | Deep | | | | |
| | | Deep | 24 | 25.4 | 5.75 | 5,851 |
| C | Hypereutrophic | Shallow | 4 | 9.8 | 6.78 | 293 |
| | | Deep | 30 | 6.2 | 6.81 | 831 |
| | | Medium | 11 | 7.8 | 6.75 | 399 |

### EMBODIMENTS

In one embodiment, the disclosure relates to a model that uses data acquired from site information, a sediment sequential phosphorus extraction, and sediment properties to build various coefficients that related to the release and/or management of phosphorus from surface water sediments. This model also calculated an overall sediment phosphorus release index that can be used to predict the release of phosphorus from the sediment sample for various time periods.

In one embodiment, the disclosure relates to a predictive sediment release feedback model that incorporates measured sediment phosphorus release rates to provide feedback to the model in order to fine tune the predictive capability and increase the accuracy of the model.

In one embodiment, the disclosure relates to a model that uses the data described in Embodiment 1 to predict the effectiveness of aluminum salts to suppress the release of sediment phosphorus into the water column.

In one embodiment, the disclosure relates to a predictive sediment release suppression feedback model that incorporates measured sediment phosphorus release suppression of various lake management phosphorus management strategies to provide feedback to the model in order to fine tune the predictive capability and increase the accuracy of the model.

In one embodiment, the disclosure relates to a method of assessing a potential of sediment in a body of water to release phosphorus into an overlaying water column, the method comprising: (a) determining site information about the body of water; (b) obtaining a sample of the sediment from the body of water; (c) determining at least one property of the sample of the sediment; (d) performing a sequential extraction on the sample of the sediment; (e) calculating at least one first sediment characteristic coefficient of the sample of the sediment, the at least one first sediment characteristic coefficient being based on at least one of the results of steps a., c., and d.; (f) calculating at least one second sediment characteristic index of the sample of the sediment, the at least one second sediment characteristic coefficient being based on at least one of the results of steps a., c., d., and e.; and (g) calculating a water column release index based at least on the results of step f. In one aspect of the embodiment, the at least one first sediment characteristic coefficient includes at least one of a sediment expansion coefficient, a sediment disturbance coefficient, and an iron stripping coefficient. In one aspect of the embodiment, the at least one first sediment characteristic index includes at least one of an oxygen depletion index, redox release index, organic phosphorus release index, and a diffusion index. In one aspect of the embodiment, the method further includes: (h) determining at least one of a measured phosphorus release over time value, the determining being based on at least one of laboratory incubations, mesocosm experiments, limnocorrals, and real-world bottom water phosphorus sampling. In one aspect of the embodiment, the determined at least one of the measured phosphorus release over time is used to provide feedback data in the method to adjust at least one of the sediment characteristic coefficients and sediment characteristic index. In one aspect of the embodiment, the method further includes: (i) estimating an effectiveness of a management strategy to suppress phosphorus release, the estimation being based at least on one of the sediment characteristic coefficients and sediment characteristic index. In one aspect of the embodiment, the method further includes (j) determining a phosphorus release suppression value, the determining being based one at least one of laboratory incubations, mesocosm experiments, limnocorrals, and real-world bottom water phosphorus sampling. In one aspect of the embodiment, the determined phosphorus release suppression value is used to provide feedback data in the method to predict effectiveness of one or more management strategies for future samples of the sediment.

What is claimed is:

1. A method for predicting total phosphorus release from sediment within a water body, the method comprising:
obtaining water body characteristics initial input data;
obtaining a sample of the sediment and obtaining, from the sample, sediment characteristics initial input data;
calculating one or more coefficients and indices based on the water body characteristics initial input data and the sediment characteristics initial input data;
calculating a predicted total phosphorus release value;
obtaining feedback input data from the water body and/or sediment; and
calculating a refined predicted total phosphorus release value based on the feedback input data.

2. The method of claim 1, further comprising:
predicting efficacy of a method of treatment of the water body based on the water body characteristics initial input data, the sediment characteristics initial input data, and/or the one or more coefficients and indices.

3. The method of claim 2, further comprising:
comparing the predicted total phosphorus release value and/or the refined predicted total phosphorus release value to a threshold predictive result value; and
determining if a method of treatment should be applied to the water body.

4. The method of claim 1, wherein the water body characteristics initial input data and the sediment characteristics initial input data include depth in a water column of the water body at which a sediment sample was collected below a surface of the water body, surface area of the water body, site-specific Osgood index, wet bulk density of the sediment, percent solids within the sediment, expanded dry bulk density of the sediment, compacted dry bulk density of the sediment, particle density of the sediment, expanded porosity of the sediment, compacted porosity of the sediment, labile organic matter content of the sediment, total organic matter content of the sediment, labile-to-total-organic matter ratio of the sediment; pH of the sediment, and/or annual pH range of bottom water of the water body.

5. The method of claim 1, wherein the step of obtaining the sediment characteristics initial input data includes performing a sediment sequential extraction to determine a total sediment phosphorus, a labile phosphorus concentration, a labile manganese concentration, a labile iron concentration, a redox-sensitive phosphorus concentration, a redox-sensitive manganese concentration, a redox-sensitive iron concentration, an organic phosphorus concentration, a metal-oxide phosphorus concentration, a metal-oxide iron concentration, a metal-oxide aluminum concentration, an aluminum-to-phosphorus ratio, a carbon-to-phosphorus ratio, an alkaline-insoluble, acid-soluble phosphorus concentration, an alkaline-insoluble, acid-soluble calcium concentration, an alkaline-insoluble, acid-soluble lanthanum concentration, an alkaline-insoluble-metal-to-phosphorus ratio, and/or a residual phosphorus concentration.

6. A computer-implemented method for predicting total phosphorus release from sediment within a water body, the method comprising:
transmitting, to a computer, water body characteristics initial input data and sediment characteristics initial input data;
performing sediment sequential extraction on a sample of the sediment from within the water body to obtain sediment extraction initial input data;
calculating, with processing circuitry of the computer, at least one sediment characteristic coefficient using the water body characteristics initial input data, the sediment characteristics initial input data, and/or the sediment extraction initial input data;
calculating, with the processing circuitry of the computer, at least one water column release index using the water body characteristics initial input data, the sediment characteristics initial input data, the sediment extraction initial input data, and/or the at least one sediment characteristic coefficient;
calculating, with the processing circuitry of the computer, a total water column release index using the at least one water column release index;
calculating, with the processing circuitry of the computer, a predicted total phosphorus release value;
transmitting to a computer feedback input data, the feedback input data including updated water body characteristics initial input data, updated sediment characteristics initial input data, and/or updated sediment extraction initial input data; and
calculating a refined predicted total phosphorus release value based on the feedback input data.

7. The computer-implemented method of claim 6, further comprising:
comparing, with the processing circuitry of the computer, the predicted total phosphorus release value and/or the refined predicted total phosphorus release value to a threshold predictive result value and determining if a method of treatment should be applied to the water body based on the comparison.

8. The computer-implemented method of claim 7, further comprising:
calculating, with the processing circuitry of the computer, a predicted aluminum binding efficiency value based on sediment.

9. The computer-implemented method of claim 8, wherein the predicted aluminum binding efficiency value is calculated based on an aluminum-to-phosphorus ratio, a metal oxide phosphorus concentration, a sediment pH, a depth in the water column at which the sediment sample was collected below a surface of the water body, and/or an annual pH range of bottom water of the water body.

10. The computer-implemented method of claim 8, wherein the method of treatment is an application of aluminum salts to the water body, the determination whether the method of treatment should be applied to the water body being based on the predicted aluminum binding efficiency value.

11. The computer-implemented method of claim 7, wherein the feedback input data includes updated water body characteristics initial input data, updated sediment characteristics initial input data, and/or updated sediment extraction initial input data that are measured after application of a method of treatment to the water body.

12. The computer-implemented method of claim 6, wherein the feedback input data includes updated water body characteristics initial input data, updated sediment characteristics initial input data, and/or updated sediment extraction initial input data that are measured from sediment incubations, mesocosms, limnocorrals, and/or real-word water column phosphorus concentrations.

13. The computer-implemented method of claim 6, wherein the water body characteristics initial input data include depth in a water column at which the sediment sample was collected below a surface of the water body, a surface area of the water body, a site-specific Osgood index;
and/or annual pH range of bottom water.

14. The computer-implemented method of claim 6, wherein the sediment characteristics initial input data include wet bulk density, percent solids, expanded dry bulk density, compacted dry bulk density, particle density, expanded porosity, compacted porosity, labile organic matter content, total organic matter content, labile-to-organic matter ratio, and/or pH.

15. The computer-implemented method of claim 6, wherein the sediment extraction initial input data include a total sediment phosphorus, a labile phosphorus concentration, a labile manganese concentration, a labile iron concentration, a redox-sensitive phosphorus concentration, a redox-sensitive manganese concentration, a redox-sensitive iron concentration, a redox-sensitive-iron-to-redox-sensitive-phosphorus ratio, an organic phosphorus concentration, a metal-oxide phosphorus concentration, a metal-oxide iron concentration, a metal-oxide aluminum concentration, aluminum-to-phosphorus ratio, a carbon-to-phosphorus ratio, an alkaline-insoluble, acid-soluble phosphorus concentration, an alkaline-insoluble, acid-soluble calcium concentration, an alkaline-insoluble, acid-soluble lanthanum concentration, an alkaline-insoluble-metal-to-phosphorus ratio, and/or a residual phosphorus concentration.

16. The computer-implemented method of claim 6, wherein the at least one sediment characteristic coefficient includes a sediment expansion coefficient, a sediment disturbance coefficient, and/or an iron stripping coefficient.

17. The computer-implemented method of claim 16, wherein the sediment expansion coefficient is calculated by the processing circuitry of the computer according to the equation:
$$\frac{Compacted dry bulk density ( \frac{g}{{cm}^{3}} )}{Expanded dry bulk density ( \frac{g}{{cm}^{3}} )} = \frac{\frac{mass of dry particles ( g )}{volume of dry particles ( {cm}^{3} )}}{Wet bulk density ( \frac{g}{{cm}^{3}} ) \times \% solids} .$$

18. The computer-implemented method of claim 6, wherein the at least one water column release initial index includes an oxygen depletion index, a diffusion index, a redox release index, and/or an organic phosphorus release index.

19. A model for predicting total phosphorus release from sediment within a water body, the predictive model comprising:
providing a database of water body characteristics initial input data obtained from a water body, a database of sediment characteristics initial input data obtained from sediment from within the water body, and a database of sediment sequential extraction initial input data obtained from sequential extraction of a sample of the sediment from within the water body;
calculating at least one sediment characteristic coefficient using the water body characteristics initial input data, the sediment characteristics initial input data, and/or the sediment extraction initial input data;
calculating at least one water column release initial index using the water body characteristics initial input data, the sediment characteristics initial input data, the sediment extraction initial input data, and/or the at least one sediment characteristic coefficient;
calculating a total water column release index using the at least one water column release index;
calculating a predicted total phosphorus release value;
obtaining feedback input data from the water body and sediment, the feedback input data including updated water body characteristics initial input data, updated sediment characteristics initial input data, and/or updated sediment extraction initial input data; and
calculating a refined predicted total phosphorus release value based on the feedback input data.

20. The model of claim 19, further comprising:
calculating a predicted aluminum binding efficiency value based on the water body characteristics initial input data, the sediment characteristics initial input data, the sediment extraction initial input data, and/or the feedback input data.
